(19) United States
(12) Patent Application Publication
(10) Pub. No.: US 2008/0254148 A1
(43) Pub. Date: Oct. 16, 2008

(54) HERBAL COMPOSITIONS FOR THE INHIBITION OF HUMAN IMMUNODEFICIENCY VIRUS

(75) Inventor: Chih-Hui Lin, Taipei City (TW)

Correspondence Address: WPAT, PC;INTELLECTUAL PROPERTY ATTORNEYS, 2030 MAIN STREET, SUITE 1300, IRVINE, CA, 92614, US

(73) Assignee: GERENT BIOTECH CO., Taipei City (TW)

(21) Appl. No.: 11/734,926

(22) Filed: Apr. 13, 2007

(51) Int. Cl. A61K 36/536; A61K 36/748; A61K 36/86; A61P 31/18

(52) U.S. Cl. 424/725

(57) ABSTRACT

The invention provides a herbal composition for the inhibition of HIV, comprising an organic extract of Hedyotis diffusa (Oldenlandia diffusa), an organic solvent extract of Prunella vulgaris and an organic solvent extract of Viola philippica (Viola yedoensis).

### FIELD OF THE INVENTION

This invention relates to herbal compositions comprising the extracts of herbs and their use in the inhibition of human immunodeficiency virus (HIV).

### BACKGROUND OF THE INVENTION

HIV (formally known as HTLV-III and lymphadenopathy-associated virus) is a retrovirus that is the cause of the disease known as AIDS (Acquired Immunodeficiency Syndrome), a syndrome where the immune system begins to fail, leading to many life-threatening opportunistic infections. HIV has been implicated as the primary cause of AIDS and can be transmitted via exposure to bodily fluids. In addition to percutaneous injury, contact with mucous membranes, non-intact skin with blood, fluids containing blood, tissue or other potentially infectious bodily fluids pose an infectious risk. Infection of human CD-4+T-lymphocytes with an HIV virus leads to depletion of this cell population, resulting in an immunodeficient state, and eventually opportunistic infections, neurological dysfunctions, neoplastic growth, and ultimately death. There are currently 18 drugs licensed and used for the treatment of HIV, and these drugs are divided into one of four classes depending on how they attack HIV. Drugs in the class of nucleoside/nucleotide reverse transcriptase inhibitors are AZT (zidovudine, Retrovir), ddI (didanosine, Videx), 3TC (lamivudine, Epivir), d4T (stavudine, Zerit), abacavir (Ziagen), and FTC (emtricitabine, Emtriva). Drugs in the class of non-nucleoside reverse transcriptase inhibitors are efavirenz (Sustiva) and nevirapine (Viramune). Drugs in the class of protease inhibitors are lopinavir/ritonavir (Kaletra), indinavir (Crixivan), ritonavir (Norvir), nelfinavir (Viracept), saquinavir hard gel capsules (Invirase), atazanavir (Reyataz), amprenavir (Agenerase), fosamprenavir (Telzir), and tipranavir (Aptivus). Only one drug is available in the class of fusion inhibitor, T20 (enfuvirtide, Fuzeon). The antiretroviral drugs are usually combined into three-drug cocktails called highly active antiretroviral therapy or HAART. However, the above-mentioned drugs still cannot effectively treat AIDS.

U.S. Pat. No. 5,837,257 provides a method for treating a subject having a viral infection selected from the group consisting of hepatitis B virus (HBV), hepatitis C virus (HCV), leukemia virus (LV) and human immunodeficiency virus (HIV), comprising administering to said subject in need thereof a therapeutically effective amount of a composition comprising a) SOLANI HERBA, prepared from the whole plant of Solanum nigrum; b) LESPEDEZAE HERBA, prepared from the whole plant of Lespedeza cuneata; c) SENECINIS HERBA, prepared from the whole plant of Senecio scandens; and d) LIGUSTRI FRUCTUS. The herbal materials of U.S. Pat. No. 5,837,257 were extracted with boiling water on a basis of 1 part by weight of plant material to approximately 5 to 10 parts by weight of water. However, the water extracts disclosed in U.S. Pat. No. 5,837,257, alone or in combination, do not contain effective anti-viral components and thus are not effective in the inhibition of HIV.

### SUMMARY OF THE INVENTION

The invention provides a herbal composition for the inhibition of HIV, comprising:

a) an organic extract of Hedyotis diffusa (Oldenlandia diffusa);

b) an organic solvent extract of Prunella vulgaris; and

c) an organic solvent extract of Viola philippica (Viola yedoensis).

### DETAILED DESCRIPTION OF THE INVENTION

The invention relates to an herbal composition for the inhibition of HIV. The present invention discovers an antiviral herbal composition produced by extracting several herbs with organic solvent. It has been found that the composition of the invention can synergistically inhibit reverse transcriptase of HIV. The composition of the invention is effective in the inhibition of HIV and is non-toxic.

The invention provides an herbal composition for the inhibition of HIV, comprising:

a) an organic extract of Hedyotis diffusa (Oldenlandia diffusa);

b) an organic solvent extract of Prunella vulgaris; and

c) an organic solvent extract of Viola philippica (Viola yedoensis).

According to the invention, the amounts of the extracts of a), b) and c) above are 15-35 wt %, 15-35 wt % and 40-60 wt %, respectively. Preferably, the amounts of the extracts of a), b) and c) above are 20-30 wt %, 20-30 wt % and 45-55 wt %, respectively. More preferably, the amounts of the extracts of a), b) and c) above are about 25 wt %, about 25 wt % and about 50 wt %, respectively

According to the invention, an “herbal composition or formulation” as used herein refers to any composition or formulation which includes herbs, herbal plants, herbal plant parts and/or herbal extracts. Thus, as used herein, an herbal composition or formulation includes herbal preparations comprising herbal food supplements, herbal medicines, herbal drugs, and medical foods. Examples of herbal compositions include, but are not limited to, the following components: a whole plant or a plant part of a single plant species; whole plants or plant parts of multiple plant species; multiple components derived from a single plant species; multiple components derived from multiple plant species; herbal extracts; or any combination of these various components. Also contemplated are herbal compositions comprising one or more chemicals derived from a single or multiple plant species.

A herbal medicine known as Hedyotis Diffusa (synonym: Oldenlandia diffusa; common name: Snake-needle grass, (also known as OLDENLANDIAE HERBA) has traditionally been used to treat illnesses such as malignant swelling, urethra infection, pharyngitis, laryngitis, tonsillitis, toxic snake bites, sub-acute or chronic coccygodynia, prurigo, carbuncle, appendicitis, intestinal cancer, contusion injuries and eye diseases; also, it reduces inflammation, relieves pain and is diuretic and antibacterial. It acts mainly on the liver and also stimulates the immune system. Hedyotis diffusa (Oldenlandia diffusa) belongs to the family Rubiaceae, which tastes sweet and is nontoxic. According to the invention, the whole plant of Hedyotis diffusa (Oldenlandia diffusa) is used to prepare the herbal compositions of the invention.

Prunella vulgaris is one of the Chinese herbs used in Asia. Prunella vulgaris L. (Labiatae) is a perennial plant known as ‘self-heal’ in Western herbal medicine. Prunella has many potential benefits, including anti-microbial, anti-viral, and anti-oxidant properties. Prunella may have activity against herpes simplex virus which could be of benefit in oral herpes and genital herpes. Prunella vulgaris and Rhizoma cibotte have shown activity in inhibiting HIV-1 (Life Sci. 2002 Aug. 30; 71(15):1779-91). Prunella vulgaris is prepared from the dried spica or whole plant of Prunella vulgaris or Prunella vulgaris subsp. The whole plant can be used as a diuretic and also has antibacterial effects in vitro. The herbal medicine tastes bitter and is nontoxic.

Viola philippica (synonym: Viola yedoensis) is known as Tokyo Violet Herb and is used to treat Carbuncles, boils, venomous snake-bites, erysipelas and other cutaneous and subcutaneous pyogenic infection. It was reported in Antiviral Res. 1988 November; 10(1-3):107-16 that the dimethylsulfoxide extract of the Chinese medicinal herb (Viola yedoensis) demonstrates high inhibitory activity toward HIV-1 in vitro and the corresponding methanol extract also showed inhibitory activity but not as much as the dimethylsulfoxide extract.

According to the invention, any suitable organic solvent can be used to extract the herbs used in the composition of the invention. Preferably, the organic solvent is selected from the group consisting of methanol, ethanol, propanol, isopropanol and acetone. More preferably, the organic solvent is selected from the group consisting of methanol, ethanol, propanol and isopropanol. Most preferably, the organic solvent is selected from the group consisting of ethanol, propanol and isopropanol.

A suitable part or parts or the whole herb of a medicinal plant was obtained and washed with cold water. The plant materials were soaked in organic solvent for over 24 hours. Preferably, the extraction is conducted at a temperature between 25^{O} C and 40^{O} C. More preferably, the extraction is conducted at about 37^{O} C. One part by weight of plant material to approximately 5 to 10 parts by weight of organic solvent was used as an extraction ratio. The extraction solution was separated from the plant material by filtration. Subsequently, the resulting solution was concentrated, and the concentrate may then be spray dried or freeze dried or absorbed by powdered material of the same plant material or starch, and thus, the single herbal medicine is prepared in powdered form. Preferably, the above-mentioned organic solvent is selected from the group consisting of methanol, ethanol, propanol, isopropanol and acetone. More preferably, the organic solvent is selected from the group consisting of methanol, ethanol, propanol and isopropanol. Most preferably, the organic solvent is selected from the group consisting of ethanol, propanol and isopropanol.

In preferred embodiments, the composition of this invention further contains a pharmaceutically acceptable carrier. As used herein, the term “pharmaceutically acceptable carrier” is meant to include one or more pharmaceutically suitable, inactive excipients, carriers, diluents, adjuvants, and lubricants. Non-limiting examples of inactive excipients, carriers, diluents, lubricants, and adjuvants which can be used in the composition of the present invention include: cellulose, substituted cellulose, calcium carbonate, dicalcium phosphate, starches, lactose, modified food starches, dextrose, calcium sulfate, magnesium carbonate, magnesium stearate, stearic acid, glycerin, vegetable oils, polysorbates, lecithin, silicon dioxide, food glaze, talc, croscarmeilose sodium, povidone, water and gelatin. Additional inactive excipients, carriers, diluents, lubricants and adjuvants which may be used with the active-ingredient composition of this invention are known in the art. The herbal compositions of the invention can be formulated into preparations in the form of a solution, tablet, capsule, powder, candy, gel or emulsion. The herbal compositions of the invention can be prepared as nutritional products or pharmaceutical products. The composition of the invention may be formulated into enteral and parenteral preparations containing an amount of the composition of the invention that is effective for inhibiting HIV. The compositions of the invention can be administered in a therapeutically effective amount of 3 to 30 grams per day.

The composition of the invention shows a synergistic effect in inhibiting reverse transcriptase of HIV. By inhibiting the reverse transcriptase of HIV, the reproduction of HIV can be successfully inhibited by the composition of the invention.

### EXAMPLE

### Inhibition of Human Immunodeficiency Virus by the Herbal Composition of the Invention

An in vitro assay of inhibiting reverse transcriptase of HIV-1 was conducted in the Institute of Medicinal Biotechnology Chinese Academy of Medical Sciences (1 Tlantan Xi Li, Beijing, 100050 P.R. China). The Hedyotis diffusa, Prunella vulgaris and Viola philippica were extracted with ethanol, respectively. The herbal composition of the invention was prepared by mixing the extracts of Hedyotis diffusa, Prunella vulgaris and Viola philippica with the ratio of 1:1:2 by weight. The resulting mixture was prepared as powders, respectively. The resulting powders were added to water and prepared as 10 mg/ml solutions. The resulting solutions were diluted to the testing solutions at different concentrations. The appropriate amounts of HIV-1 reverse transcriptase were added to the 60 ul reaction mediums containing 50 mmol/L of Tris-HCL (pH 7.8), 6 mmol/L of MgCl₂, 100 mmol/L of KCl, 0.133 mg/ml bovine serum albumin (BSA), 2 ug/ml of rA.(dT)₁₅ and 0.2 umol/L of ³HdTTP. Each of the resulting solutions of Hedyotis diffusa, Prunella vulgaris and Viola philippica and the herbal composition of the invention were added to the reaction mediums at 37^{O} C for 30 minutes to determine the counts per minute (cpm) by Scintillation Counter (Backman LS 6000). The IC₅₀ values of Hedyotis diffusa, Prunella vulgari, Viola philippica and the herbal composition of the invention were calculated according to Reed & Muench method (Reed L J, Muench H. Aourmal of simple method of estimating fifty percent endpoints. Am J Hyg, 1938, 27(3):493-497) and the results thereof were listed in below table:

| | IC₅₀ of the herbal composition |
|---|---|
| Testing sample | of the invention (μg/ml) |
| Hedyotis diffusa | — |
| Prunella vulgari | — |
| Viola philippica | 475.05 |
| Herbal composition of the invention | 60.97 |
| (Hedyotis diffusa + Prunella vulgari + | |
| Viola philippica) | |

As shown in the table above, Hedyotis diffusa and Prunella vulgari do not have effect in inhibiting HIV-1 reverse transcriptase. The IC50 value of the herbal composition of the invention against HIV-1 reverse transcriptase is much lower than that of Viola philippica. Given the above, in comparison with Hedyotis diffusa, Prunella vulgari or Viola philippica per se the herbal composition of the invention has a synergistic effect in inhibiting HIV.

What is claimed is:

1. A herbal composition for the inhibition of HIV, comprising:
a) an organic extract of Hedyotis diffusa (Oldenlandia diffusa);
b) an organic solvent extract of Prunella vulgaris; and
c) an organic solvent extract of Viola philippica (Viola yedoensis).

2. The herbal composition of claim 1, wherein the amounts of the extracts of a), b) and c) are 15-35 wt %, 15-35 wt % and 40-60 wt %, respectively.

3. The herbal composition of claim 1, wherein the amounts of the extracts of a), b) and c) are 20-30 wt %, 20-30 wt % and 45-55 wt %, respectively.

4. The herbal composition of claim 1, wherein the amounts of the extracts of a), b) and c) are about 25 wt %, about 25 wt % and about 50 wt %, respectively.

5. The herbal composition of claim 1, wherein the organic solvent is selected from the group consisting of methanol, ethanol, propanol, isopropanol and acetone.

6. The herbal composition of claim 1, wherein the organic solvent is selected from the group consisting of methanol, ethanol, propanol and isopropanol.

7. The herbal composition of claim 1, wherein the organic solvent is selected from the group consisting of ethanol, propanol and isopropanol.

8. The herbal composition of claim 1, wherein the organic solvent is ethanol.

9. The herbal composition of claim 1, which has a therapeutically effective amount of 3 to 30 grams per day.

10. The herbal composition of claim 1, which can be further formulated in a beverage, capsule, tablet, powder, candy, gel, nutritional product or pharmaceutical product.
